(19) United States
(12) Patent Application Publication
(10) Pub. No.: US 2026/0166014 A1
(43) Pub. Date: Jun. 18, 2026

(54) X842 FORMULATION

(71) Applicants: GUIZHOU SINORDA BIOMEDICINE CO., LTD., Guiyang (CN); SHANGHAI SINORDA BIOMEDICINE CO., LTD., Shanghai (CN)

(72) Inventors: Jianxin WANG, Shanghai (CN); Jiangang YANG, Shanghai (CN); Pingsheng HU, Guiyang (CN); Ming LU, Guiyang (CN)

(21) Appl. No.: 18/851,965

(22) PCT Filed: Mar. 23, 2023
(86) PCT No.: PCT/CN2023/083375

§ 371 Date: Sep. 27, 2024

(30) Foreign Application Priority Data
Mar. 30, 2022 (WO) PCT/CN2022/084214

(51) Int. Cl. A61K 31/437; A61K 9/20

(52) U.S. Cl. CPC A61K 31/437; A61K 9/2009; A61K 9/2018; A61K 9/2027; A61K 9/2031; A61K 9/2054

(57) ABSTRACT

The present disclosure provides a pharmaceutical formulation for oral administration comprising a solid dispersion comprising amorphous X842 and a water-soluble, amphiphilic carrier.

### TECHNICAL FIELD

The present disclosure relates to the field of treatment of a gastrointestinal inflammatory disease or a gastric acid related disease and in particular a solid dispersion (SD) of X842 developed for this purpose.

### BACKGROUND

WO 2010/063876 discloses that the compound 5-{2-[({8-[(2,6-dimethylbenzyl)amino]-2,3-dimethylimidazo[1,2-a]pyridine-6-yl}carbonyl)-amino]ethoxy}-5-oxopentanoic acid, which is often referred to as X842, is an effective inhibitor of gastric secretion.

X842 has the following formula:

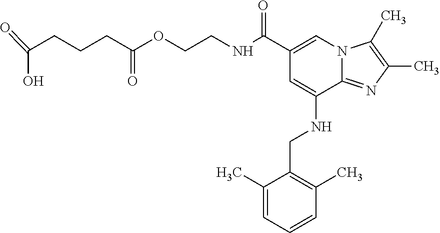 [SMILES: Cc1cccc(C)c1CNc1cc(C(=O)NCCOC(=O)CCCC(=O)O)cn2c(C)c(C)nc12]

US2022002297 discloses two crystalline forms of X842 and states that for use in pharmaceutical preparations, it is desirable that the active pharmaceutical ingredient (API) is in a highly crystalline form.

The solubility of X842 is however very low, which makes is difficult to prepare an effective oral formulation of X842.

### SUMMARY

It is an objective of the present disclosure to overcome X842's solubility problem and thereby facilitate effective treatments using the drug.

The present inventors have found that this objective is met by a solid dispersion of X842 and a carrier, in which solid dispersion X842 has an amorphous form.

Accordingly, the present disclosure provides the following itemized listing of embodiments.
1. A pharmaceutical formulation for oral administration comprising a solid dispersion comprising amorphous X842 and a water-soluble, amphiphilic carrier.
2. The pharmaceutical formulation of item 1, wherein the carrier is polymeric carrier.
3. The pharmaceutical formulation of item 2, wherein the polymeric carrier comprises vinylcaprolactam.
4. The pharmaceutical formulation of item 2 or 3, wherein the polymeric carrier comprises vinyl acetate.
5. The pharmaceutical formulation of any one of items 2-4, wherein the average molecular weight of the polymeric carrier is in the range of 40,000-250,000 g/mol when determined by gel permeation chromatography.
6. The pharmaceutical formulation of item 5, wherein the average molecular weight of the polymeric carrier is in the range of 80,000-150,000 g/mol when determined by gel permeation chromatography.
7. The pharmaceutical formulation of any one of the preceding items, wherein the dry weight ratio of X842 to the carrier is between 2:1 and 1:20, such as between 1:2 and 1:10, such as between 1:1 and 1:10, such as between 1:2 and 1:7, such as between 1:3 and 1:7, such as between 1:3 and 1:5.
8. The pharmaceutical formulation of any one of the preceding items, wherein the solid dispersion is formed by spray drying.
9. The pharmaceutical formulation of item 8, wherein the solid dispersion is obtained by spray drying a solution of X842 and the carrier.
10. The pharmaceutical formulation of item 9, wherein the solid dispersion is obtained by spray drying a solution of X842 and the carrier in organic solvent. The organic solvent is any solvent that is suitable for spray drying X842, and is preferably absolute ethanol, methanol, acetone, tetrahydrofuran, dimethyl sulfoxide and chloroform.
11. The pharmaceutical formulation of any one of the preceding items, which is in the form of a unit dose.
12. The pharmaceutical formulation of item 11, wherein the amount of amorphous X842 in the unit dose is 10-100 mg, such as 10-60 mg, such as 10-40 mg, such as 10-25 mg, such as 10-24 mg.
13. The pharmaceutical formulation of any one of items 1-12 for use in a method of treatment of a gastrointestinal inflammatory disease or a gastric acid related disease, such as erosive gastroesophageal reflux disease (eGERD).
14. A solid dispersion comprising amorphous X842 and a water-soluble, amphiphilic carrier, wherein said solid dispersion is obtainable by spray drying a solution of X842 and the carrier.
15. The solid dispersion of item 14, wherein the solution is a solution of X842 and the carrier in organic solvent. The organic solvent is any solvent that is suitable for spray drying X842, and is preferably absolute ethanol, methanol, acetone, tetrahydrofuran, dimethyl sulfoxide and chloroform.
16. The solid dispersion of item 14 or 15, wherein the nozzle temperature is in the range of 55-99° C., such as in the range of 65-95° C., such as in the range of 70-85° C. during said spray drying.
17. The solid dispersion of any of items 14-16, wherein the temperature of the solution being fed to the nozzle during spray drying is in the range of 25-99° C., such as 55-99° C., such as in the range of 65-95° C., such as in the range of 70-85° C.
18. The pharmaceutical formulation of any one of items 1-13 or the solid dispersion of any one of items 14-17, which has a shelf life of 3 months or longer, preferably 6 months or longer, more preferably 12 months or longer.
19. The pharmaceutical formulation of any one of items 1-13 or the solid dispersion of any one of items 14-18, the oral bioavailability of which is higher than that of the crystalline X842.
20. The pharmaceutical formulation of any one of items 1-13 or the solid dispersion of any one of items 14-18, the oral bioavailability such as AUC or Cₘₐₓ of which is 1.2 times or more, 1.5 times or more, twice or more of that of the crystalline X842, preferably three times or more, more preferably 6 times or more of that of the crystalline X842.
21. The solid dispersion of any one of items 14-20 for use in a method of treatment of a gastrointestinal inflammatory disease or a gastric acid related disease, such as erosive gastroesophageal reflux disease (eGERD).
22. A tablet or capsule comprising the pharmaceutical formulation of any one of items 1-13 and 18-20 or the solid dispersion of any one of items 14-20.
23. The tablet or capsule of item 22, further comprising a disintegrant, such as polyvinylpolypyrrolidone.
24. The tablet or capsule of item 22 or 23, wherein the dry weight ratio of disintegrant to X842 is at least 1.1:1, such as at least 1.4:1, such as at least 1.7:1, such as at least 2:1, such as at least 2.8:1.
25. The tablet or capsule of any one of items 22-24, further comprising a cellulosic excipient, such as microcrystalline cellulose (MCC).
26. The tablet or capsule of any one of items 22-25, further comprising lactose.
27. The tablet or capsule of any one of items 22-26, wherein the tablet or capsule has a shelf life of 3 months or longer, preferably 6 months or longer, more preferably 12 months or longer.
28. The tablet or capsule of any one of items 22-27, the oral bioavailability of which is higher than that of the X842 capsule comprising the crystalline X842.
29. The tablet or capsule of any one of items 22-28, the oral bioavailability such as AUC or Cₘₐₓ of which is 1.2 times or more, 1.5 times or more, twice or more of that of the X842 capsule, preferably three times or more, more preferably 6 times or more of that of the X842 capsule comprising the crystalline X842.
30. Use of the pharmaceutical formulation of any one of items 1-13 and 18-20 in preparation of a medicament for treating a gastrointestinal inflammatory disease or a gastric acid related disease, such as erosive gastroesophageal reflux disease (eGERD).
31. Use of the solid dispersion of any one of items 14-20 in preparation of a medicament for treating a gastrointestinal inflammatory disease or a gastric acid related disease, such as erosive gastroesophageal reflux disease (eGERD).

### BRIEF DESCRIPTION OF THE DRAWINGS

FIG. 1 shows the solubility of X842 in mixtures or solid dispersions (formed by the solvent method) with different carriers in different mass ratios (see Example 1 below).

FIG. 2 shows X842 solubility measurement results for different solid dispersions and physical mixtures. “Sol” means Soluplus.

FIG. 3 shows PXRD diffractograms for three different solid dispersions (one prepared by spray drying, one prepared by hot melt extrusion (HME) and one prepared by the solvent method), a physical mixture, Soluplus and X842.

FIG. 4 and FIG. 5 show PXRD diffractograms for Samples 3.1-3.11 (the preparations of these samples are described in the Examples section below).

FIG. 6 and FIG. 7 show PXRD diffractograms for Samples 3.12-3.18 (the preparations of these samples are described in the Examples section below).

FIG. 8 shows X842 solubility measurement results for Sample 3.12, 3.13, 3.16, 3.17 (solid dispersions formed by spray drying), Sample 3.15 and 3.18 (physical mixture).

FIG. 9 shows the X842 dissolution from tablets formed from Compositions 1-4 (the preparations of these tablets is described in the Examples section below) and from a reference capsule.

FIG. 10 shows the stability experiment result that solid dispersion powder placed 6 months at room temperature.

FIG. 11 shows the stability experiment result that solid dispersion tablet is placed at room temperature for 3 months.

FIG. 12 and FIG. 13 show the pharmacokinetic parameter result of solid dispersion tablet and reference capsule in the beagle dog body.

### DETAILED DESCRIPTION

In the context of the present invention, “shelf life” means that the life period of a drug wherein 10% or less of the drug is degraded.

In the context of the present invention, “crystalline X842” means a state in which X842 exists in a stable crystalline form, in contrast to the amorphous state, such “crystalline X842” is for example Forms A and B as disclosed in US2022002297A1, especially Form A.

As a first aspect of the present disclosure, there is provided a pharmaceutical formulation for oral administration. The formulation comprises a solid dispersion comprising amorphous X842 and a water-soluble, amphiphilic carrier. This formulation facilitates quick dissolution of X842 and may thus be referred to as an immediate release pharmaceutical formulation.

The carrier is typically polymeric. In one embodiment, the polymeric carrier comprises vinylcaprolactam and/or vinyl acetate. Preferably, the polymeric carrier comprises vinylcaprolactam and vinyl acetate.

The average molecular weight (as determined by gel permeation chromatography) of the polymeric carrier is preferably in the range of 40,000-250,000 g/mol, more preferably in the range of 80,000-150,000 g/mol.

An example of a polymeric carrier that comprises vinylcaprolactam and vinyl acetate and has an average molecular weight (as determined by gel permeation chromatography) in the range of 80,000-150,000 g/mol is Soluplus from BASF.

The dry weight ratio of X842 to the carrier is preferably between 2:1 and 1:20, preferably between 1:1 and 1:10, more preferably between 1:1 and 1:10, more preferably between 1:2 and 1:7, more preferably between 1:3 and 1:7, most preferably between 1:3 and 1:5.

In an embodiment of the first aspect, the solid dispersion is formed by spray drying. Typically, the solid dispersion is obtained by spray drying a solution of X842 and the carrier. The solution may for example be an ethanol solution. Further embodiments of the spray drying are discussed below in connection to the third aspect.

The pharmaceutical formulation of the first aspect may for example be in the form of a unit dose. In such a unit dose, the amount of amorphous X842 may be 10-100 mg, such as 10-60 mg, such as 10-40 mg, such as 10-25 mg, such as 10-24 mg. The efficient dissolution of X842 means that the amount of X842 may be relatively low.

As a second aspect of the present disclosure, the pharmaceutical formulation of the first aspect is provided for use in a method of treatment of a gastrointestinal inflammatory disease or a gastric acid related disease, such as erosive gastroesophageal reflux disease (eGERD). In the method of treatment, the pharmaceutical formulation is intended to be administrated orally.

As a third aspect of the present disclosure, there is provided a solid dispersion comprising amorphous X842 and a water-soluble, amphiphilic carrier, wherein said solid dispersion is obtainable by spray drying a solution, such as an ethanol solution, of X842 and the carrier.

The nozzle temperature may for example be in the range of 55-99° C., such as in the range of 65-95° C., such as in the range of 70-85° C. during said spray drying. Further, the temperature of the solution being fed to the nozzle during said spray drying may be in the range of 25-99° C., such as in the range of 55-99° C., such as in the range of 65-95° C., such as in the range of 70-85° C. When the solution is an ethanol solution, the nozzle temperature and the temperature of the solution being fed to the nozzle are preferably in the range of 65-78° C., more preferably in the range of 65-75° C.

Otherwise, the embodiments and examples of the first aspect apply to the third aspect mutatis mutandis.

As a fourth aspect of the present disclosure, the solid dispersion of the third aspect is provided for use in a method of treatment of a gastrointestinal inflammatory disease or a gastric acid related disease, such as eGERD. In the method of treatment, the solid dispersion is intended to be administrated orally.

As a fifth aspect of the present disclosure, there is provided a tablet or capsule comprising the pharmaceutical formulation of the first aspect or the solid dispersion of the third aspect.

The tablet or capsule may further comprise a disintegrant, such as a non-cellulosic disintegrant. A preferred example of a non-cellulosic disintegrant is polyvinylpolypyrrolidone. The dry weight ratio of disintegrant to X842 is typically at least 1.1:1, such as at least 1.4:1, preferably at least 1.7:1, such as at least 2:1, such as at least 2.8:1. An upper limit for this dry weight ratio may be 4:1.

In one embodiment, the tablet or capsule further comprises a cellulosic excipient and/or lactose. The cellulosic excipient may for example be MCC.

The amount of amorphous X842 in the tablet or capsule may be 10-100 mg, such as 10-60 mg, such as 10-40 mg, such as 10-25 mg, such as 10-24 mg.

As a sixth aspect of the present disclosure, the tablet or capsule of the fifth aspect is provided for use in a method of treatment of a gastrointestinal inflammatory disease or a gastric acid related disease, such as eGERD. In the method of treatment, the tablet or capsule is intended to be administrated orally.

The subject of the therapeutic methods discussed above is preferably a human.

As a seventh aspect of the present disclosure, there is provided use of the pharmaceutical formulation of first aspect or the solid dispersion of the third aspect or the tablet or capsule of the fifth aspect for preparation of the medicament for treating a gastrointestinal inflammatory disease or a gastric acid related disease, such as erosive gastroesophageal reflux disease (eGERD).

As an eighth aspect of the present disclosure, after the solid dispersion powder of the third aspect is placed at room temperature for 3 months or longer, preferably 6 months or longer, more preferably 12 months or longer, its PXRD shows that it is still amorphous and remains stable.

As a ninth aspect of the present disclosure, after the solid dispersion tablet or capsule of the fifth aspect is placed at room temperature for 3 months or longer, preferably 6 months or longer, more preferably 12 months or longer, its dissolution rate is basically the same as before, and it remains stable.

As a tenth aspect of the present disclosure, after the solid dispersion tablet or capsule of the fifth aspect are administered orally, the oral bioavailability thereof is higher than that of the X842 capsule comprising the crystalline X842, such as AUC or Cₘₐₓ. For example, the oral bioavailability of the solid dispersion tablet or capsule of the fifth aspect is 1.2 times or more, twice or more of that of the X842 capsule, preferably three times or more, more preferably 6 times or more of that of the X842 capsule. For example, the AUC of the solid dispersion tablet or capsule of the fifth aspect is 1.2 times or more, twice or more of that of the X842 capsule, preferably three times or more, more preferably 6 times or more of that of the X842 capsule. For example, the Cₘₐₓ of the solid dispersion tablet or capsule of the fifth aspect is 1.2 times or more, twice or more of that of the X842 capsule, preferably three times or more, more preferably 6 times or more of that of the X842 capsule.

### EXAMPLES

### Example 1

Solid dispersions of X842 and different carriers (in different ratios) were prepared by the solvent method, and their solubility in pH 6.8 was determined.

X842 (API) and carrier was added to a 250 ml beaker and about 150 mL of anhydrous ethanol was then added. The amount of API was about 70 mg and the API to carrier dry weight ratio was 1:3, 1:5 or 1:10. The carrier was PVP/VA64 (poly-(vinylpyrrolidone-co-vinylacetate) from BASF), PVP K30 (Polyvinylpyrrolidone K30 ((C₆H₉NO)ₙ) from BASF), Affinisol-15 LV (HPMC), Plasdone™ s-630 (a 60:40 linear random copolymer of N-vinyl-2-pyrrolidone and vinyl acetate), PEG6000, Soluplus (a polyvinyl caprolactam-polyvinyl acetate-polyethylene glycol graft co-polymer (PEG 6000:vinylcaprolactam:vinyl acetate=13:57:30) from BASF) or Klucel EF (hydroxypropylcellulose (HPC)). Ultrasonication was used to completely dissolve the API in the ethanol.

The solution was added to porcelain dish and placed in an 80° C. water bath to evaporate the solvent. The solid residue was then placed in a vacuum drying oven at 40° C. for 24 h. The dried residue was ground finely through a 65 mesh sieve

As references, physical mixtures of the API and each of the carriers in a 1:10 dry weight ratio were prepared.

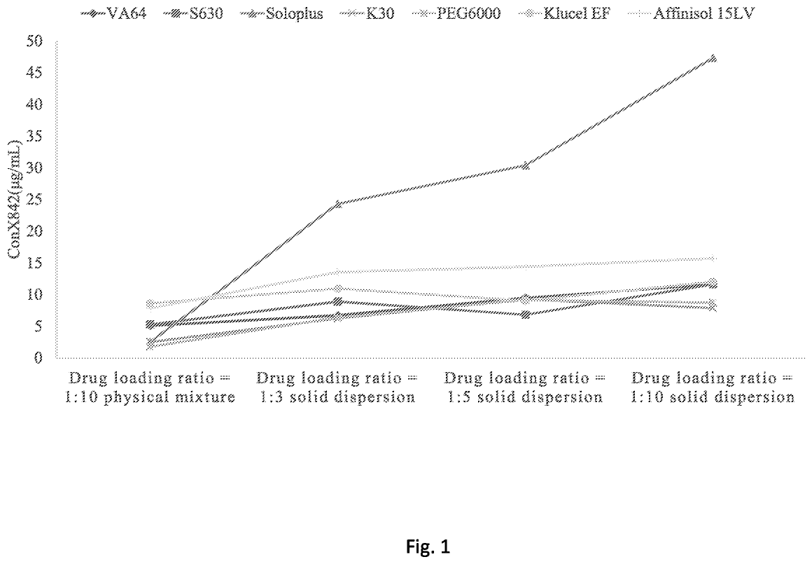

The API solubility for each of the solid dispersions and mixtures was measured according to the following protocol. Solid dispersion/mixture was added to a 50 mL centrifuge tube and 20 mL of pH 6.80 phosphate buffer was added followed by shaking (shaking speed: 130 r/min) in a shaker for 24 h at 25° C. An appropriate amount of liquid was then taken from the tube and was passed through a 0.22 μm microporous membrane. Finally, HPLC was used to determine the X842 solubility (n=3). The results are presented in table 1 below and in FIG. 1.

TABLE 1
The X842 solubility (μg/mL) in phosphate buffer (pH 6.80)
of various physical mixtures (M) and solid dispersions (SD).

| | PVP/ | | | PVP | | Klucel | Affinisol- |
|---|---|---|---|---|---|---|---|
| | VA64 | s-630 | Soluplus | K30 | PEG6000 | EF | 15LV |
| M, 1:10 ratio | 5.08 | 5.26 | 2.52 | 2.49 | 1.76 | 8.60 | 7.82 |
| SD, 1:3 ratio | 6.73 | 8.88 | 24.29 | 6.21 | 6.38 | 10.92 | 13.52 |
| SD, 1:5 ratio | 9.49 | 6.81 | 30.38 | 9.25 | 9.14 | 9.00 | 14.40 |
| SD, 1:10 ratio | 11.58 | 11.59 | 47.28 | 7.83 | 8.69 | 12.02 | 15.70 |

Notably, table 1 shows that the Soluplus carrier is ineffective in physical mixture with the API (also in comparison to most of the other carriers), but outperforms all the other carriers in a solid dispersion independent of the ratio (see also FIG. 1).

### Example 2.1

A solid dispersion was prepared from X842 (17 wt. %) and Soluplus (83 wt. %) according to the following:

1 g of X842 and 5 g of Soluplus were mixed well to get a physical mixture. A hot melt extruder was opened, and the extrusion temperature was set to 160° C. (twin screw speed of 100 rpm). When the temperature was reached and after stabilization for half an hour, the physical mixture was hot melt extruded to obtain extruded strips. The hot melt extruded strips were cooled and crushed through a 65 mesh sieve to obtain a solid dispersion.

The solid dispersion was analyzed using powder X-ray diffraction (PXRD). Further, the X842 solubility of the solid dispersion was tested. This is described below.

### Example 2.2

A solid dispersion was prepared from X842 (17 wt. %) and Soluplus (83 wt. %) according to the following:

1 g of X842 and 5 g of Soluplus were mixed well to get a physical mixture. A hot melt extruder was opened, and the extrusion temperature was set to 120° C. (twin screw speed of 100 rpm). When the temperature was reached and after stabilization for half an hour, the physical mixture was hot melt extruded to obtain extruded strips. The hot melt extruded strips were cooled and crushed through a 65 mesh sieve to obtain a solid dispersion.

The X842 solubility of the solid dispersion was tested. This is described below.

### Example 2.3

A solid dispersion was prepared from X842 (17 wt. %), polymer carrier polyvinyl alcohol (66 wt. %) and sorbitol (17 wt. %) according to the following:

1 g of X842, 4 g of polyvinyl alcohol (PVOH) and 1 g of sorbitol were mixed well to get a physical mixture. A hot melt extruder was opened, and the extrusion temperature was set to 190° C. (twin screw speed of 75 rpm). When the temperature was reached and after stabilization for half an hour, the physical mixture was hot melt extruded to obtain extruded strips. The hot melt extruded strips were cooled and crushed through a 65 mesh sieve to obtain a solid dispersion.

The X842 solubility of the solid dispersion was tested. This is described below.

### Example 2.4

A solid dispersion was prepared from X842 (17 wt. %), polymer carrier polyvinyl alcohol (41.5 wt. %) and sorbitol (41.5 wt. %) according to the following:

1 g of X842, 4 g of polyvinyl alcohol (PVOH) and 1 g of sorbitol were mixed well to get a physical mixture. A hot melt extruder was opened, and the extrusion temperature was set to 190° C. (twin screw speed of 75 rpm). When the temperature was reached and after stabilization for half an hour, the physical mixture was hot melt extruded to obtain extruded strips. The hot melt extruded strips were cooled and crushed through a 65 mesh sieve to obtain a solid dispersion.

The X842 solubility of the solid dispersion was tested. This is described below.

### Example 2.5

A solid dispersion was prepared from X842 (17 wt. %) and Soluplus (83 wt. %) using spray drying according to the following:

0.5 g of X842 and 2.5 g Soluplus were added to anhydrous ethanol, which was heated to 70° C. in a water bath to completely dissolve the X842. A spray drying equipment was opened, and the nozzle temperature was set to 75° C. When the temperature was reached, the solution was pumped into the nozzle by a peristaltic pump (5-20 rpm) for spray drying.

The solid dispersion was analyzed using powder X-ray diffraction (PXRD). Further, the X842 solubility of the solid dispersion was tested. This is described below.

### Example 2.6 (Solubility Determination)

The X842 solubility of the solid dispersions prepared in Examples 2.1-2.5 and a X842/Soluplus solid dispersion formed by the solvent method (1:5 dry weight ratio, see Example 1) was tested. Physical mixtures of X842 and carrier was also tested as references.

25 mg (measured by X842) of solid dispersion or physical mixture was added to 25 mL pH 6.8 phosphate buffer to prepare a suspension containing about 1 mg/mL X842. The suspension was shaken in a shaker (shaking speed: 130 rpm). After 1, 2, 4, 8 and 24 hours a 2 mL sample was taken and filtered through a 0.22 μm microporous membrane. Methanol was added to achieve a dilution of 2 or 10 times (to reach a concentration within the range of determination). 10 μL was injected into HPLC to determine the solubility.

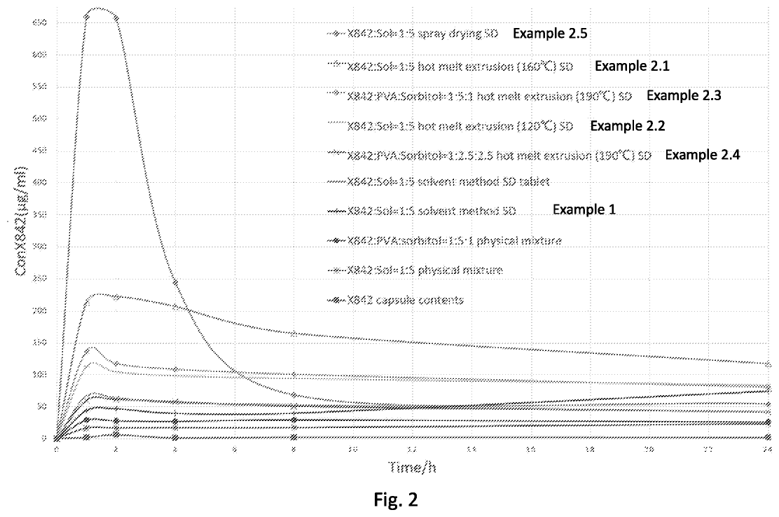

The results are presented in FIG. 2, which shows that the solid dispersion prepared according to Example 2.5 is significantly better than the other solid dispersions and the physical mixtures.

### Example 2.7 (PXRD)

PXRD was carried out to compare the solid dispersion of Example 2.5 (which has superior X842 solubility) to the solid dispersion of Example 2.1 (prepared by hot melt extrusion at 160° C.). Further, PXRD was carried out on a X842/Soluplus solid dispersion formed by the solvent method (1:5 dry weight ratio, see Example 1), a X842/Soluplus physical mixture (1:5 dry weight ratio), Soluplus alone and X842 alone were also analyzed to provide further references.

For the PXRD experiments, the instrument was a Nippon Science Smartlab9 kw and the conditions were as follows: Cu target, Kα radiation (λ=0.15406 nm), operating voltage and current of 45 kV and 100 mA, respectively, and scanned at a rate of 4°/min in the range of 20 from 5 to 40°.

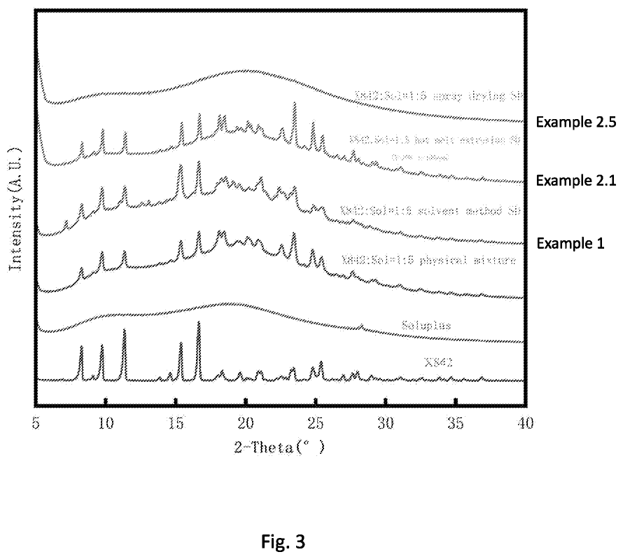

The resulting PXRD diffractograms are presented in FIG. 3, which shows that the crystallinity of X842 is retained in the solid dispersions formed by hot melt extrusion and the solvent method and in the physical mixture. Further, FIG. 2 shows that these forms have inferior X842 solubility compared to the solid dispersion formed by spray drying (Example 5.2). Finally, FIG. 3 shows that X842 is amorphous in the solid dispersion formed by spray drying. FIGS. 2 and 3 thus show that a solid dispersion in which X842 is amorphous is key to X842's solubility.

### Example 3.1 (PXRD)

As second round of PXRD was carried out. This round included the following samples:
3.1—Spray-dried (nozzle temp=75° C.) solid dispersion of X842 and Soluplus in a 1:3 ratio;
3.2—Solid dispersion of Example 2.5;
3.3—Soluplus only;
3.4—Hot melt-extruded (T=175° C., 50 rpm) solid dispersion of X842 and Soluplus in a 1:5 ratio;
3.5—Hot melt-extruded (T=175° C., 50 rpm) solid dispersion of X842, Soluplus and mannitol in a 1:3:2 ratio;
3.6—Hot melt-extruded (T=160° C., 30 rpm) solid dispersion of X842 and PVP/VA64 in a 1:5 ratio;
3.7—Physical mixture of X842 and Soluplus in a 1:5 ratio;
3.8—X842 only;
3.9—Spray-dried (nozzle temp=60° C., ethanol) solid dispersion of X842 and Soluplus in a 1:3 ratio;
3.10—Spray-dried (nozzle temp=90° C., ethanol) solid dispersion of X842 and Soluplus in a 1:3 ratio; and
3.11—Spray-dried (nozzle temp=75° C., methanol) solid dispersion of X842 and Soluplus in a 1:3 ratio.

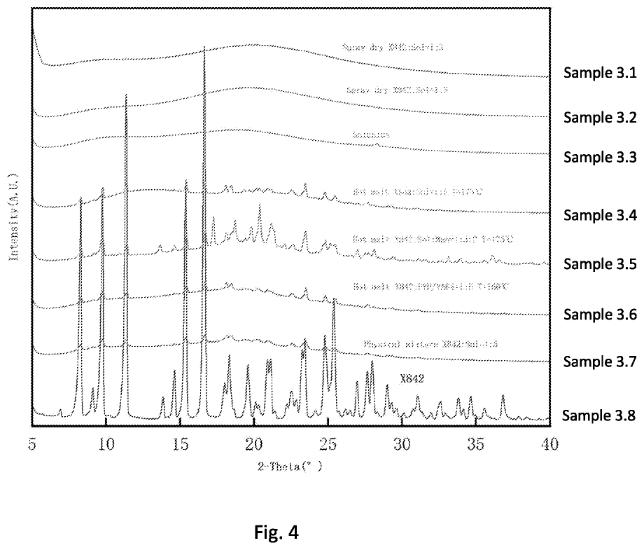

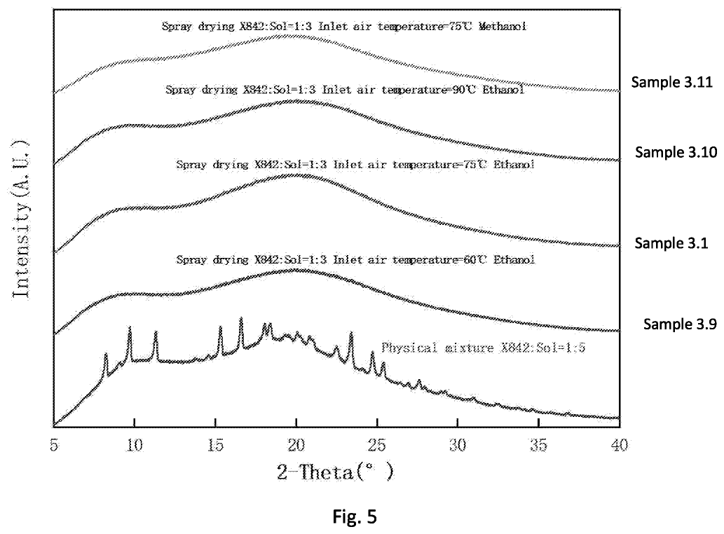

The resulting PXRD diffractograms are presented in FIGS. 4 and 5, which again shows that spray drying of a X842/Soluplus solution produce a solid dispersion comprising amorphous X842, whereas the hot melt-extruded solid dispersion comprises crystalline X842.

### Example 3.2 (PXRD)

As third round of PXRD was carried out. This round included the following samples:
3.12—Spray-dried (nozzle temp=75° C.) solid dispersion of X842 and Soluplus in a 1:3 ratio;
3.13—Spray-dried (nozzle temp=75° C.) solid dispersion of X842, Soluplus and SiO₂ in a 1:1:1 ratio;
3.14—SiO₂ only;
3.15—Physical mixture of X842, Soluplus and SiO₂ in a 1:1:1 ratio;
3.16—Spray-dried (nozzle temp=75° C.) solid dispersion of X842 and Soluplus in a 1:1 ratio; and
3.17—Spray-dried (nozzle temp=75° C.) solid dispersion of X842 and Soluplus in a 1:5 ratio;
3.18—Physical mixture of X842 and Soluplus in a 1:5 ratio.

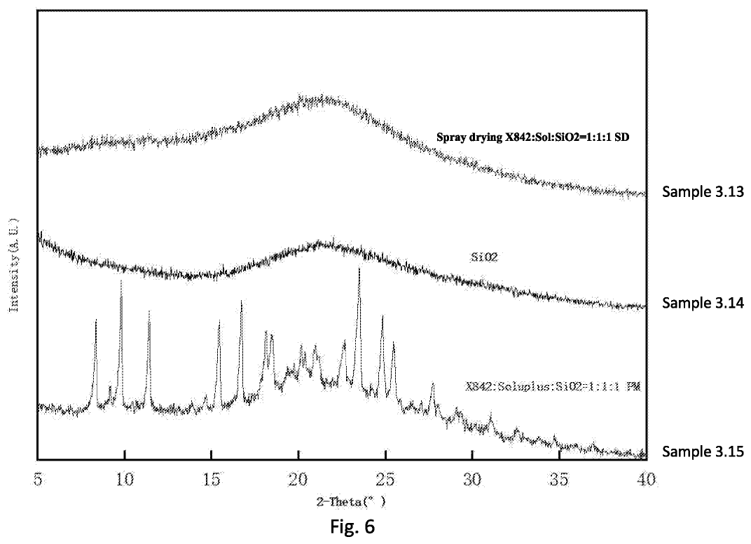

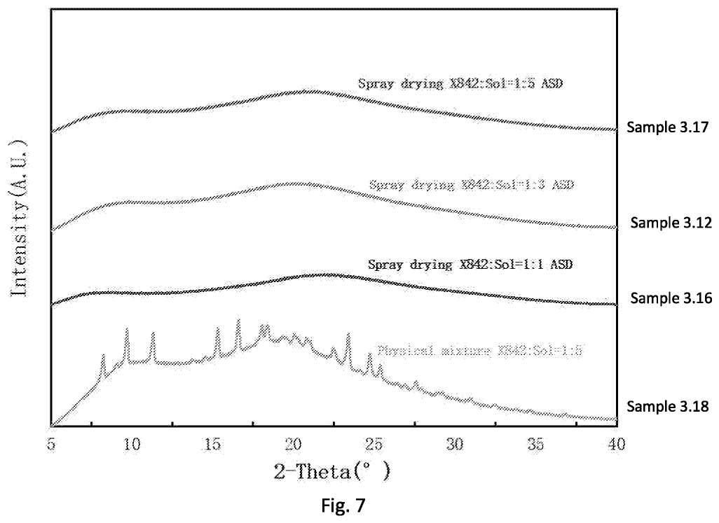

The resulting PXRD diffractograms are presented in FIG. 6 and FIG. 7, which again shows that spray drying of a X842/Soluplus solution produce a solid dispersion comprising amorphous X842, not only in a 1:5 ratio, but also in 1:3 and 1:1 ratios.

### Example 3.3 (Solubility Determination)

The X842 solubility of samples 3.12, 3.13, 3.15, 3.16 and 3.17 was measured according to the following:

25 mg (measured by X842) of sample material was added to 25 mL pH 6.8 phosphate buffer to prepare a suspension containing about 1 mg/mL X842. The suspension was shaken in a shaker (shaker shaking speed=130 rpm; temperature=25° C.). After 1, 2, 4, 8 and 24 hours a 2 mL suspension sample was taken and filtered through a 0.22 μm microporous membrane. To 0.5 mL of the filtrate, methanol was added to achieve a dilution of 10 or 2 times (to reach a concentration within the range of determination). 10 μL was injected into HPLC to determine the solubility.

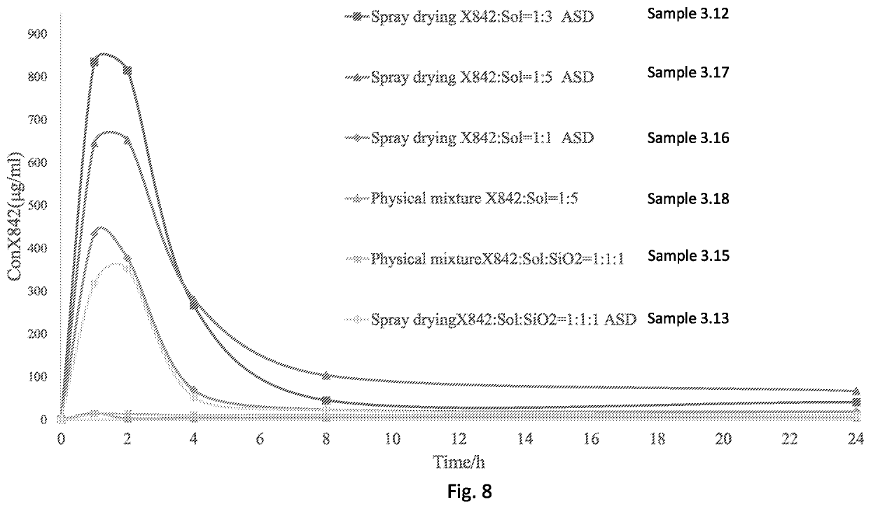

The results are presented in FIG. 8, which shows that the solid dispersions comprising amorphous X842 have drastically improved X842 solubility compared to a physical mixture comprising crystalline X842. FIG. 8 further shows that an X842 to Soluplus ratio of 1:5 or 1:3 is more effective than a X842 to Soluplus ratio of 1:1 and that out of the ratios tested, both 1:3 and 1:5 are very effective.

### Example 4.1 (Preparation of Tablets)

Tablets were prepared from compositions comprising various solid dispersions of X842 and Soluplus prepared by spray drying or hot-melt extrusion. The weight of each tablet was 350 mg and the amount of X842 in each tablet was 25 mg.

Composition 1, prepared by the following method: the solid dispersion (prepared by spray drying), MicroceLac 100 (microcrystalline fibers and lactose in a 1:3 ratio), and silicon dioxide were placed in a self-sealing bag and mixed well and then directly tableted (tablet thickness=0.8-1.0 mm; tableting pressure=4-6 kN; tablet hardness is 60-80 N).

| | Weight per | |
|---|---|---|
| | tablet (mg) | Proportion |
| Solid dispersion | 105(X842: 25 mg) | 30% |
| (X842:Soluplus ≈ 1:3) | | |
| MicroceLac 100 | 203 | 58% |
| Polyvinylpolypyrrolidone | 35 | 10% |
| (PVPP) | | |
| Silicon dioxide | 7 | 2% |
| Total | 350 | 100% |

Composition 2, prepared by the following method: the solid dispersion (prepared by hot melt extrusion), MicroceLac 100 (microcrystalline cellulose and lactose in a 1:3 ratio), and silicon dioxide were placed in a self-sealing bag and mixed well and then directly tableted (tableting pressure=18-20 kN; tablet hardness is 60-70 N).

| | Weight per | |
|---|---|---|
| | tablet (mg) | Proportion |
| Solid dispersion | 172(X842: 25 mg) | 49% |
| (X842:Soluplus ≈ 1:5) | | |
| MicroceLac 100 | 136 | 39% |
| Polyvinylpolypyrrolidone | 35 | 10% |
| Silicon dioxide | 7 | 2% |
| Total | 350 | 100% |

Composition 3, prepared by the following method: the solid dispersion (prepared by spray drying), MicroceLac 100 (microcrystalline cellulose and lactose in a 1:3 ratio), and silicon dioxide were placed in a self-sealing bag and mixed well and then directly tableted (tablet thickness=0.8-1.0 mm; tableting pressure=4-6 kN; tablet hardness is 60-80 N).

| | Weight per | |
|---|---|---|
| | tablet (mg) | Proportion |
| Solid dispersion | 75 (X842: 25 mg) | 21% |
| (X842:Soluplus:SiO₂ = 1:1:1) | | |
| MicroceLac 100 | 198 | 57% |
| Polyvinylpolypyrrolidone | 70 | 20% |
| Silicon dioxide | 7 | 2% |
| Total | 350 | 100% |

Composition 4, prepared by the following method: the solid dispersion (prepared by spray drying), MicroceLac 100 (microcrystalline cellulose and lactose in a 1:3 ratio), and silicon dioxide were placed in a self-sealing bag and mixed well and then directly tableted (tablet thickness=0.5-0.8 mm; tableting pressure=5-8 kN; tablet hardness is 50-80 N).

| | Weight per | |
|---|---|---|
| | tablet (mg) | Proportion |
| Solid dispersion | 105(X842: 25 mg) | 30% |
| (X842:Soluplus ≈ 1:3) | | |
| MicroceLac 100 | 168 | 48% |
| Polyvinylpolypyrrolidone | 70 | 20% |
| Silicon dioxide | 7 | 2% |
| Total | 350 | 100% |

### Example 4.2 (Dissolution Determination)

According to the dissolution degree determination method (Chinese Pharmacopoeia 2020 edition of the four general principles 0931 second method paddle method), two tablets (corresponding to about 50 mg of X842) for each composition were placed in a cup and 900 mL of pH 6.8 phosphate buffer (dissolution medium) was added to the cup to prepare a suspension. The suspension was shaken in a shaker (shaker shaking speed=100 rpm). After 0.5, 1, 2, 4, 6, 8 and 10 hours a 5 mL suspension sample was taken (while supplementing the same volume of media) and filtered through a 0.22 μm microporous membrane. The filtrate was diluted two times with methanol and then filtered again and injected into HPLC to determine the solubility by measuring the peak area.

As a reference, capsules containing a composition comprising crystalline X842 (such as Form A as disclosed in US2022002297) was suspended and analyzed in the same way.

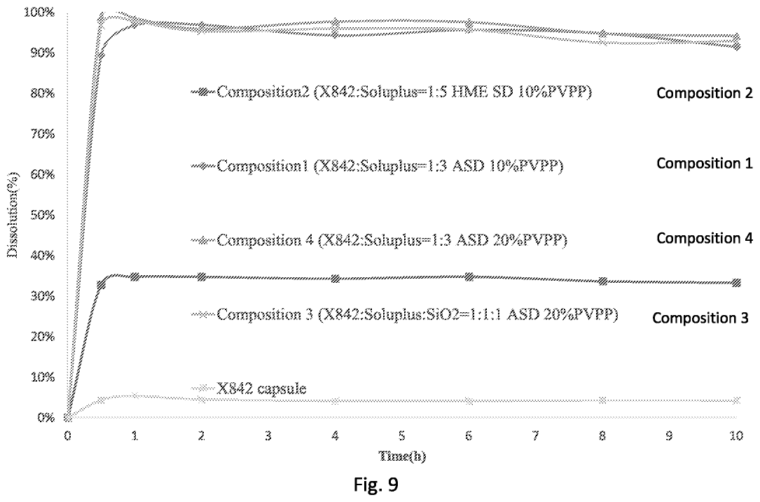

The resulting dissolution of X842 is presented in Table 2 below and FIG. 9, which show that the tablets comprising a solid dispersion of amorphous X842 (i.e. the tablets formed from compositions 1, 3 and 4) outperformed the tablet comprising a solid dispersion of crystalline X842 (composition 2) and the X842 capsule reference (also comprising crystalline X842).

TABLE 2
X842 SD Tablet Test-Average Value Based on 3
Parallel Tests, Dissolution Medium: pH 6.8

| Test sample | 0 h | 0.5 h | 1 h | 2 h | 4 h | 6 h | 8 h | 10 h |
|---|---|---|---|---|---|---|---|---|
| Composition 2 (X842:Soluplus = 1:5 | 0% | 33% | 35% | 35% | 34% | 35% | 34% | 33% |
| HME SD 10% PVPP) | | | | | | | | |
| Composition 1 (X842:Soluplus = 1:3 | 0% | 89% | 97% | 97% | 94% | 96% | 95% | 91% |
| ASD 10% PVPP) | | | | | | | | |
| Composition 4 (X842:Soluplus = 1:3 | 0% | 99% | 98% | 96% | 98% | 98% | 95% | 94% |
| ASD 20% PVPP) | | | | | | | | |
| Composition 3 | 0% | 97% | 98% | 95% | 96% | 96% | 92% | 93% |
| (X842:Soluplus:SiO2 = 1:1:1 ASD | | | | | | | | |
| 20% PVPP) | | | | | | | | |
| X842 capsule | 0% | 4% | 5% | 5% | 4% | 4% | 4% | 4% |

### Example 5 (Stability Test of the Solid Dispersion Powder)

The X842 ASD powder prepared by spray drying method is placed in ziplock bag, is stored at room temperature for about 6 months, and its PXRD figure is collected with the previous method.

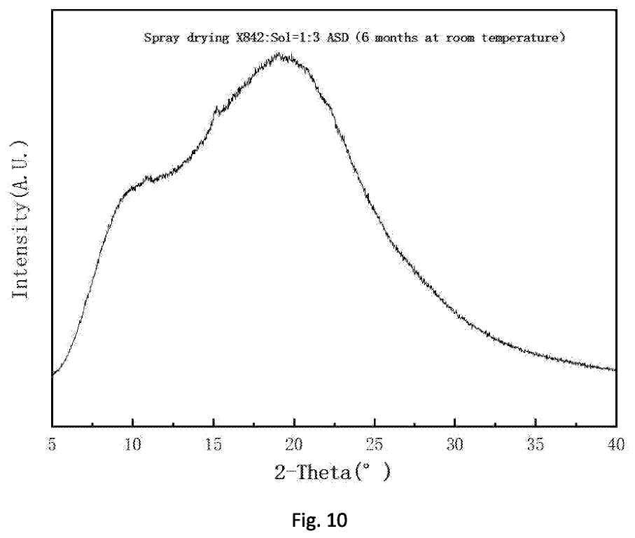

The PXRD figure of the X842 ASD powder is as shown in FIG. 10, which shows that the X842 ASD powder has good stability and is still amorphous after being stored at room temperature for about 6 months.

### Example 6 (Stability Test of the Solid Dispersion Tablet)

With the afore-mentioned prepared tablet comprising the solid dispersion: composition 1 and composition 2, after being stored at room temperature for at least 3 months, the dissolution stability of the tablets after storing was measured according to the dissolution determination method as mentioned in Example 4.2.

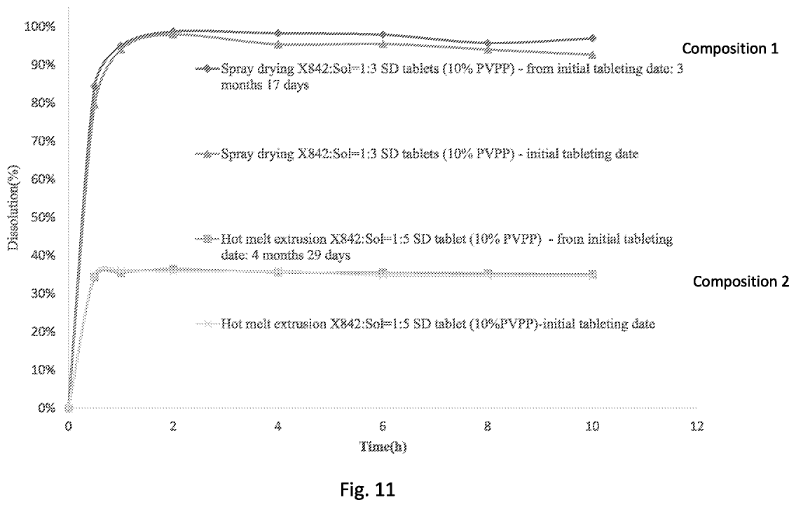

The dissolution stability test result of described tablets is as shown in table 3 below and FIG. 11, which show that dissolution rate after said storing is basically the same as that at the initial tableting date, indicating that the tested tablets have good stability.

TABLE 3
X842 SD Tablet Stability Test- Average Value Based
on 3 Parallel Tests, Dissolution Medium: pH 6.8

| Test sample | 0 | 0.5 | 1 | 2 | 4 | 6 | 8 | 10 |
|---|---|---|---|---|---|---|---|---|
| Spray drying X842:Sol = 1:3 SD tablets | 0% | 84% | 95% | 99% | 98% | 98% | 96% | 97% |
| (10% PVPP) - from initial tableting | | | | | | | | |
| date: 3 months 17 days | | | | | | | | |
| Spray drying X842:Sol = 1:3 SD tablets | 0% | 79% | 94% | 98% | 95% | 95% | 94% | 93% |
| (10% PVPP) - initial tableting date | | | | | | | | |
| Hot melt extrusion X842:Sol = 1:5 SD | 0% | 34% | 35% | 36% | 36% | 36% | 35% | 35% |
| tablet (10% PVPP) - from initial | | | | | | | | |
| tableting date: 4 months 29 days | | | | | | | | |
| Hot melt extrusion X842:Sol = 1:5 SD | 0% | 35% | 36% | 36% | 36% | 35% | 35% | 35% |
| tablet (10% PVPP)-initial tableting | | | | | | | | |
| date | | | | | | | | |

### Example 7 (In Vivo Pharmacokinetic Study)

Experimental method: 6 Beagle dogs with the body weight between 8 kg-12 kg are used for the test after being raised for 1 week in a standard animal breeding room. After being fasted for 24 hours before the test, they were divided into two groups; a single-dose self-crossover test was adopted, and the dosage was 50 mg/dog; a total of 4 groups including X842 spray drying excipient physical mixture, X842 spray drying ASD (Composition 1) tablet, X842 hot melt extruded SD (Composition 2) and X842 capsules, are administered, with a washout period of 7 days for each group. 1.5 mL of blood was collected at 11 points before administration and 0.25 h, 0.5 h, 1 h, 2 h, 3 h, 4 h, 6 h, 8 h, 12 h and 24 h after administration. The blood is centrifuged at 5000 rpm for 10 minutes, the upper layer of plasma was separated at 2-8° C., and the collected plasma samples were stored in a −80° C. refrigerator before analysis. After analysis, the remaining plasma samples are continuously stored in a −80° C. refrigerator. After taking out the plasma sample, put it at room temperature, vortex and mix well (if necessary, the blank sample can be centrifuged before use), put 80 μL sample into a 1.5 mL centrifuge tube, add 40 μL methanol and 360 μL internal standard solution (for the blank sample, add the same volume of methanol instead of the internal standard), vortex to mix, centrifuge for 5 minutes (14000 rpm), take a certain amount of supernatant, and conduct LC-MS/MS sample analysis. Analyze and record the peak areas for X842, the active metabolite TX07 (8-[(2,6-dimethylbenzyl)amino]-N-(3-hydroxypropyl)-2,3-dimethylimidazo[1,2-a]pyridine-6-carboxamide, Linaprazan, AZDo865) and internal standard, and use DAS software for data processing.

Use the following chromatographic conditions: model for the chromatographic column: Thermo AQUASIL C18, 4.6 μm, 50 mm×2.1 mm; Mobile phase: A is 0.1% formic acid aqueous solution, B is 0.1% formic acid methanol solution; Needle washing liquid: 50% methanol water; Mobile phase gradient Elution procedure is shown in the following table:

| Time (min) | 0.5 | 2.5 | 2.51 | 3.5 |
|---|---|---|---|---|
| B phase(%) | 40 | 95 | 40 | Stop |

Initial gradient (B %): 40%; column temperature: 40° C.; flow rate: 0.6 mL/min; injection volume: 3 μL; run time: 3.5 min; autosampler temperature: 4° C.

Mass Spectrometry Conditions: LCMS-8060 Mass Spectrometer with ESI Source, Positive Ion MRM Scanning

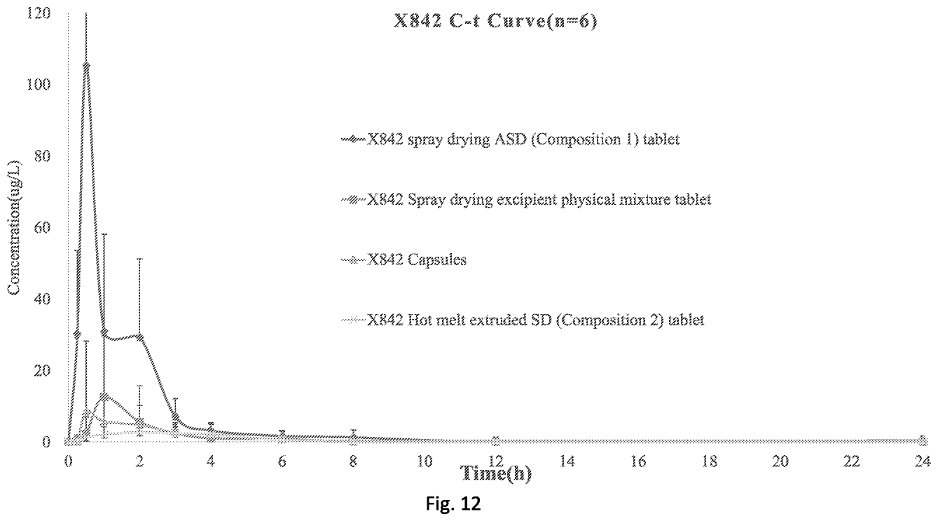

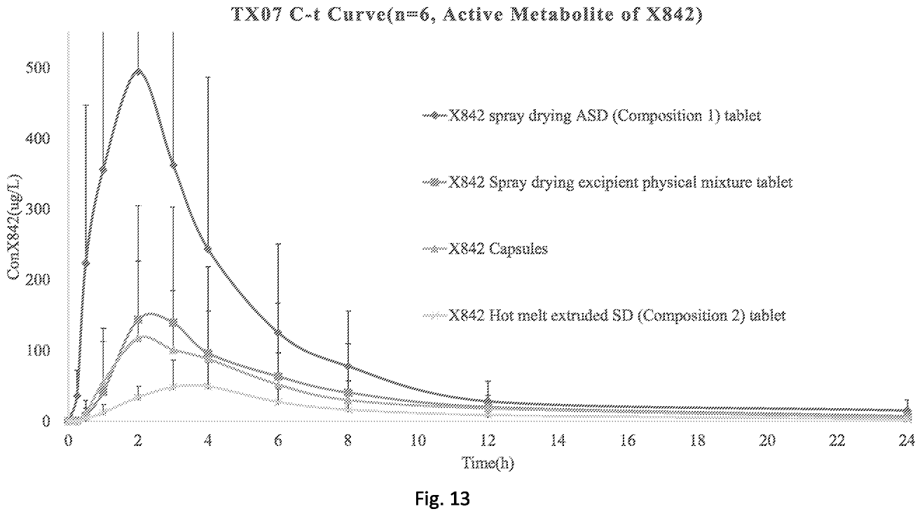

As the experimental results, the plasma concentration-time curves for the 4 groups including the X842 spray drying excipient physical mixture, X842 spray drying ASD (Composition 1) tablet, X842 hot melt extruded SD (Composition 2) and X842 capsules are shown in FIGS. 12 and 13, and some PK parameters are shown in Table 3 and Table 4.

TABLE 3
X842 PK parameters

| | | X842 spray | X842 spray | | X842 hot melt |
|---|---|---|---|---|---|
| | | drying ASD | drying excipient | | extruded SD |
| | | (Composition 1) | physical mixture | X842 | (Composition 2) |
| Parameters | Unit | tablet | tablet | capsules | tablet |
| T_{1/2α} | h | 46.27 | 0.15 | 11.66 | 11.88 |
| T_{1/2β} | h | 46.33 | 0.39 | 23.27 | 15.63 |
| Tₘₐₓ | h | 0.50 | 1.00 | 0.50 | 2.00 |
| Cₘₐₓ | ng/mL | 105.39 | 12.59 | 8.65 | 2.74 |
| AUC₍₀₋₂₄₎ | ng/mL*h | 269.61 | 4.99 | 34.69 | 7.95 |
| AUC_{(0-∞)} | ng/mL*h | 403.94 | 5.96 | 45.65 | 13.29 |

TABLE 4
TX07 PK parameters (Active metabolite of X842)

| | | X842 spray | X842 spray | | X842 hot melt |
|---|---|---|---|---|---|
| | | drying ASD | drying excipient | | extruded SD |
| | | (Composition 1) | physical mixture | X842 | (Composition 2) |
| Parameters | Unit | tablet | tablet | capsules | tablet |
| T_{1/2α} | h | 1.47 | 1.38 | 1.47 | 2.11 |
| T_{1/2β} | h | 19.81 | 37.63 | 19.50 | 37.04 |
| Tₘₐₓ | h | 2.00 | 2.00 | 2.00 | 4.00 |
| Cₘₐₓ | ng/mL | 494.50 | 143.97 | 117.45 | 49.17 |
| AUC₍₀₋₂₄₎ | ng/mL*h | 2422.70 | 900.72 | 745.29 | 342.42 |
| AUC_{(0-∞)} | ng/mL*h | 2512.56 | 940.62 | 777.33 | 356.87 |

According to the prototype drug X842 measurement results as shown in Table 3, the AUC (0-24 h) for the X842 spray drying ASD (Composition 1) tablet group is maximum at 269.61 ng/mL*h; the X842 capsule group is at 34.69/mL*h; the X842 hot melt extruded SD (Composition 2) tablet group is 7.95 ng/ml*h; the X842 spray drying excipient physical mixture tablet group is the smallest at 4.99 ng/mL*h; the X842 spray drying ASD (Composition 1) tablet group is 7.77, 33.91 and 54.03 times of the other three groups respectively.

According to the analysis results for active metabolite TX07 as shown in Table 4: the AUC (0-24 h) for the X842 spray drying ASD (Composition 1) tablet group is the largest at 2422.70 ng/ml*h; the X842 spray drying excipient physical mixture tablet group is 900.72 g/mL*h; the X842 capsule group is 745.29 ng/ml*h; the X842 hot melt extruded SD (Composition 2) tablet group is minimum at 342.42 ng/ml*h; the X842 spray drying ASD (Composition 1) tablet group is 2.39, 3.25 and 7.08 times of the other three groups respectively.

The above results show that of both X842 and the active metabolite TX07 are measured at much higher concentrations for the X842 spray drying ASD group, indicating that the bioavailability thereof is significantly improved compared with the other groups.

The blood concentration of the X842 solid dispersion (spray drying method) tablet of the present invention is higher than the X842 capsule in the body of beagle dog, proving that the X842 amorphous solid dispersion in the present invention can better maintain supersaturated concentration of X842 in the stomach and the intestinal tract, thereby increasing its oral bioavailability.

Each technical characteristic of the above-mentioned examples can be combined arbitrarily, for making description succinct, all possible combinations of each technical characteristic in the above-mentioned example are not all described, yet, as long as the combination of these technical characteristics does not results in a contradiction, all should be deemed to be within the scope of this invention.

The above-mentioned examples have only expressed several implementation modes of the present invention, and its description is comparatively specific and detailed, but can not therefore be interpreted as the restriction to invention patent scope. It should be pointed out that those skilled in the art can make several modifications and improvements without departing from the concept of the present invention, and these all belong to the protection scope of the present invention. Therefore, the protection scope of the patent for the present invention should be based on the appended claims.

1. A pharmaceutical formulation for oral administration comprising a solid dispersion comprising amorphous X842 and a water-soluble, amphiphilic carrier.

2. The pharmaceutical formulation of claim 1, wherein the carrier is polymeric carrier.

3. The pharmaceutical formulation of claim 2, wherein the polymeric carrier comprises vinylcaprolactam.

4. The pharmaceutical formulation of claim 2, wherein the polymeric carrier comprises vinyl acetate.

5. The pharmaceutical formulation of claim 2, wherein the average molecular weight of the polymeric carrier is in the range of 40,000-250,000 g/mol, preferably 80,000-150,000 g/mol, when determined by gel permeation chromatography.

6. The pharmaceutical formulation of claim 1, wherein the dry weight ratio of X842 to the carrier is between 2:1 and 1:20, such as between 1:1 and 1:10, such as between 1:2 and 1:10, such as between 1:2 and 1:7, such as between 1:3 and 1:7, such as between 1:3 and 1:5.

7. The pharmaceutical formulation of claim 1, wherein the solid dispersion is formed by spray drying.

8. The pharmaceutical formulation claim 1, which is in the form of a unit dose.

9. The pharmaceutical formulation of claim 8, wherein the amount of amorphous X842 in the unit dose is 10-100 mg, such as 10-60 mg, such as 10-40 mg, such as 10-25 mg, such as 10-24 mg.

10. The pharmaceutical formulation of claim 1, which has a shelf life of 3 months or longer, preferably 6 months or longer, more preferably 12 months or longer.

11. The pharmaceutical formulation of claim 1, the oral bioavailability such as AUC or Cₘₐₓ of which is higher than that of the crystalline X842, such as 1.2 times or more, 1.5 times or more, twice or more of that of the crystalline X842, preferably three times or more, more preferably 6 times or more of that of the crystalline X842.

12. The pharmaceutical formulation claim 1 for use in a method of treatment of a gastrointestinal inflammatory disease or a gastric acid related disease, such as erosive gastroesophageal reflux disease (eGERD).

13. A solid dispersion comprising amorphous X842 and a water-soluble, amphiphilic carrier, wherein said solid dispersion is obtainable by spray drying a solution of X842 and the carrier, such as an ethanol solution of X842 and the carrier.

14. The solid dispersion of claim 13 for use in a method of treatment of a gastrointestinal inflammatory disease or a gastric acid related disease, such as erosive gastroesophageal reflux disease (eGERD).

15. A tablet or capsule comprising the pharmaceutical formulation of any one of claim 1.

16. The tablet of claim 15, further comprising a disintegrant, such as polyvinylpolypyrrolidone.

17. The tablet or capsule of claim 15, wherein the dry weight ratio of disintegrant to X842 is at least 1.1:1, such as at least 1.4:1, such as at least 1.7:1, such as at least 2:1, such as at least 2.8:1.

18. A tablet or capsule comprising the solid dispersion of any one of claim 13.

19. The tablet of claim 18, further comprising a disintegrant, such as polyvinylpolypyrrolidone.

20. The tablet or capsule of claim 18, wherein the dry weight ratio of disintegrant to X842 is at least 1.1:1, such as at least 1.4:1, such as at least 1.7:1, such as at least 2:1, such as at least 2.8:1.
